(19) United States
(12) Patent Application Publication
(10) Pub. No.: US 2024/0197500 A1
(43) Pub. Date: Jun. 20, 2024

(54) Fabric Reinforced Prosthetic Suspension Sleeve

(71) Applicant: Alps South, LLC, St. Petersburg, FL (US)

(72) Inventors: Aldo Laghi, Pinellas Park, FL (US); Nathaniel Vint, Palm Harbor, FL (US); David Carson, Clearwater, FL (US); Jakob W. McClelland, Gulfport, FL (US)

(21) Appl. No.: 18/542,839

(22) Filed: Dec. 18, 2023

Related U.S. Application Data
(60) Provisional application No. 63/463,129, filed on May 1, 2023.
(60) Provisional application No. 63/433,946, filed on Dec. 20, 2022.

(51) Int. Cl. A61F 2/78

(52) U.S. Cl. CPC A61F 2/7812; A61F 2002/7837

(57) ABSTRACT

A prosthetic suspension sleeve with a fabric section of 5-60 degrees that allows for a greater comfort for the wearer. The use of polymeric material is easier on the skin while also enhancing the function of the sleeve by retaining its location due to the grip on the skin and exterior socket wall and sealing the socket and wearer's skin to allow a vacuum to form or be created within the socket. The sleeve generally comprises an internal gel or silicone layer and an outer fabric layer, both of which are pre-flexed to a 5 to 60-degree bend at about a midsection of the sleeve, and an internal fabric layer which is located throughout the midsection of the suspension sleeve depending on the placement of a prosthetic socket. The prosthetic suspension sleeve may also be a straight sleeve with fabric reinforcement. The internal fabric layer may alternatively further comprise an internal layer of sprayed silicone.

### CROSS-REFERENCE TO RELATED INVENTIONS

This Application claims priority to U.S. Provisional Patent Application No. 63/433,946, filed on Dec. 20, 2022, and titled “Fabric Reinforced Pre-Flexed Prosthetic Suspension Sleeve”, and to U.S. Provisional Patent Application No. 63/463,129, filed on May 1, 2023, and titled “Fabric Reinforced Pre-Flexed Prosthetic Suspension Sleeve,” the contents of which are incorporated by reference herein in their entirety.

### BACKGROUND OF THE INVENTION

### Field of the Invention

The described invention relates to prosthetic suspension sleeves. Specifically, the described invention relates to a fabric-reinforced pre-flexed prosthetic suspension sleeve incorporating an internal fabric section designed to protect the suspension sleeve against the wear and tear of prosthetic sockets.

### Description of the Background Art

Prosthetic suspension sleeves serve the purpose of maintaining a seal between a residual limb and a socket. Various means and apparatus have been developed with the goal of providing a secure attachment that provides the amputee with the ability to conduct his or her affairs without fear of losing the prosthesis during normal or strenuous activities. Some more active amputees, in some extreme circumstances, will resort to using duct tape to seal holes in order to keep the suspension sleeve from migrating in a direction during long distance activities such as marathons. Many of these devices do not solve the issue of positioning, wherein the residual limb moves within the socket of the prosthesis in response to forces exerted on the prosthesis. Pistoning further promotes tissue and skin problems including but not limited to irritation and lesions.

The main challenge with polymeric prosthetic suspension sleeves is the fragility of the polymeric material when compared to a socket wall. Use of polymeric material is easier on the skin while also enhancing the function of the sleeve by retaining its location due to the grip on the skin and exterior socket wall and sealing the socket and wearer's skin to allow a vacuum to form or be created within the socket. When impacted, the polymeric material will be the first to fail due to the socket wall cutting through the sleeve and allowing the vacuum to fail. Alternatively, the sleeve can wear at the location of the socket edge due to increased stretch and stress while flexing the knee during a normal stride. This stress is only amplified when high activity is involved. A pre-flex of the prosthetic sleeve allows for a reduction in these stresses by allowing the material to be at a resting state mid stride but will still see peak forces through the stride along the socket brim. Protection and reinforcement of the sleeve is required to increase the lifecycle and comfort for the wearer of the sleeve while still providing the acceptable functionality of a prosthetic sleeve.

Therefore, it is an object of this invention to provide an improvement which overcomes the aforementioned inadequacies of the prior art devices and provides an improvement which is a significant contribution to the advancement of the prosthetic suspension sleeve art.

Another object of the invention is to provide a prosthetic suspension sleeve that prevents or minimizes damage due to the socket striking an object and causing it to cut the sleeve.

Another object of the invention is to provide a prosthetic suspension sleeve that allows that sleeve to slide or traverse over the edge of the socket brim reducing the forces applied to the polymeric sleeve and reducing the likelihood of losing suction.

Another object of the invention is to provide a prosthetic suspension sleeve that increases comfort for the user.

Another object of the invention is to provide a prosthetic suspension sleeve that increases the life cycle of the sleeve.

Another object of the invention is to provide a prosthetic suspension sleeve that reduces stress in the tensile direction during the mid-swing of the gait cycle.

Another object of the invention is to provide a prosthetic suspension sleeve that reduces bunching behind the knee during the mid-swing of the gait cycle.

Another object of the invention is to provide a prosthetic suspension sleeve that allows for movement around the socket brim without migration of the sleeve during use.

Another object of the invention is to provide a prosthetic suspension sleeve that greatly reduces the friction between the exterior socket wall and the sleeve allowing the polymeric sleeve to slide slightly over the edge of the socket brim rather than causing the polymeric material to stretch excessively and cause wear.

Another object of the invention is to provide a pre-flexed prosthetic suspension sleeve that comprises uniform film of an interior elastomer/adhesive layer on the internal fabric so as to create further frictional forces between the socket and the sleeve as well as to encourage vacuum between the limb and socket while still allowing for the sleeve to stretch when donned.

The foregoing has outlined some of the pertinent objects of the invention. These objects should be construed to be merely illustrative of some of the more prominent features and applications of the intended invention. Many other beneficial results can be attained by applying the disclosed invention in a different manner or modifying the invention within the scope of the disclosure. Accordingly, other objects and a fuller understanding of the invention may be had by referring to the summary of the invention and the detailed description of the preferred embodiment in addition to the scope of the invention defined by the claims taken in conjunction with the accompanying drawings.

### SUMMARY OF THE INVENTION

The present invention relates generally to a prosthetic suspension sleeve with a fabric section of 5-60 degrees that allows for a greater comfort for the wearer. The use of polymeric material is easier on the skin while also enhancing the function of the sleeve by retaining its location due to the grip on the skin and exterior socket wall and sealing the socket and wearer's skin to allow a vacuum to form or be created within the socket. The sleeve generally comprises an internal gel or silicone layer and an outer fabric layer, both of which are pre-flexed to a 5 to 60-degree bend at about a midsection of the sleeve, and an internal fabric layer which is located throughout the midsection of the suspension sleeve depending on the placement of a prosthetic socket. The prosthetic suspension sleeve may also be a straight sleeve with fabric reinforcement. The internal fabric layer may alternatively further comprise an internal layer of sprayed silicone.

The foregoing has outlined rather broadly the more pertinent and important features of the present invention in order that the detailed description of the invention that follows may be better understood so that the present contribution to the art can be more fully appreciated. Additional features of the invention will be described hereinafter which form the subject of the claims of the invention. It should be appreciated by those skilled in the art that the conception and the specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims.

### BRIEF DESCRIPTION OF THE DRAWINGS

For a more complete understanding of the present disclosure and its advantages, reference is now made to the following descriptions, taken in conjunction with the accompanying drawings, in which:

FIG. 1 is a side perspective of a limb system comprising a limb, cushion liner, and socket without a sleeve;

FIG. 2 is a cross-sectional view showing the sleeve of the present invention;

FIG. 3 is a cross-sectional view showing use of the sleeve of the present invention with the limb system shown in FIG. 1;

FIG. 4 is a perspective view of the sleeve of the present invention highlighting the pre-flexed bend;

FIG. 5 is a top perspective view of the present invention;

FIG. 6 is a cross-sectional view of the present invention along line J-J of FIG. 5;

FIG. 7 is a cross-sectional view of an alternative embodiment of the present invention as a straight sleeve;

FIG. 8 is a cross-sectional view of an alternative embodiment of the present invention utilizing longitudinal retention strips and circumferential bands;

FIG. 9 is a cross-sectional view of an alternative embodiment of the present invention utilizing circumferential bands;

FIG. 10 is a cross-sectional view of an alternative embodiment of the present invention utilizing longitudinal retention strips;

FIG. 11A is a perspective view of an alternative embodiment of the present invention;

FIG. 11B is a cross-sectional view of FIG. 11A along line M-M;

FIG. 12A is a perspective view of an alternative embodiment of the present invention; and

FIG. 12B is a cross-sectional view of FIG. 12A along line O-O.

Similar reference numerals refer to similar parts throughout the several views of the drawings.

### DETAILED DESCRIPTION OF THE PREFERRED EMBODIMENT

The following description is of the best mode presently contemplated for carrying out the invention. This description is not to be taken in a limiting sense, but is made merely for the purpose of describing one or more preferred embodiments of the invention. The scope of the invention should be determined with reference to the claims.

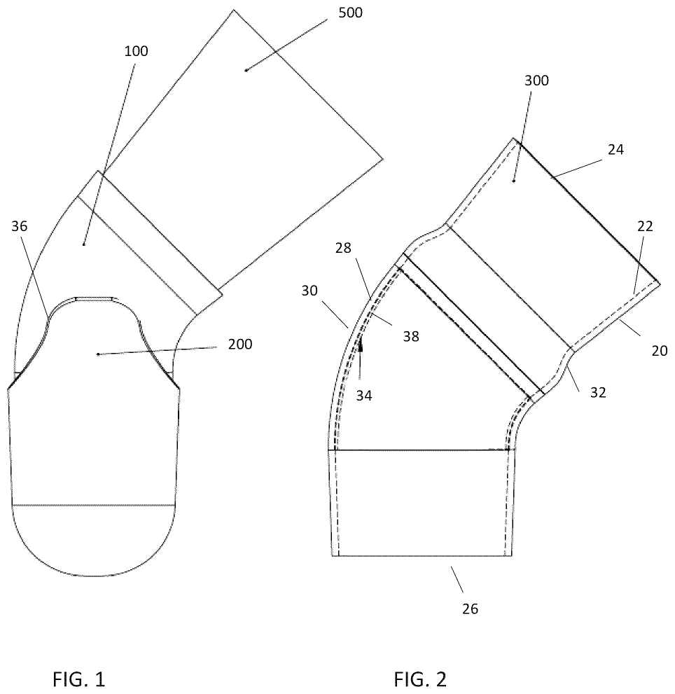

As shown in FIG. 1, prosthetic users first insert their residual limb 500 into a cushion liner 100 after which the user inserts the residual limb and liner 100 into a socket 200 having a brim 36. Liner 100 comprises an open proximal end and a closed distal end as well as an external fabric layer and an internal gel or silicone layer. Socket 200 is made of a hard plastic or carbon fiber resin and comprises a patellar opening, a pair of raised ears, and a rear opening. Prior art methods then involved adding a suspension sleeve over the socket/liner/limb combination in order to create an airtight seal.

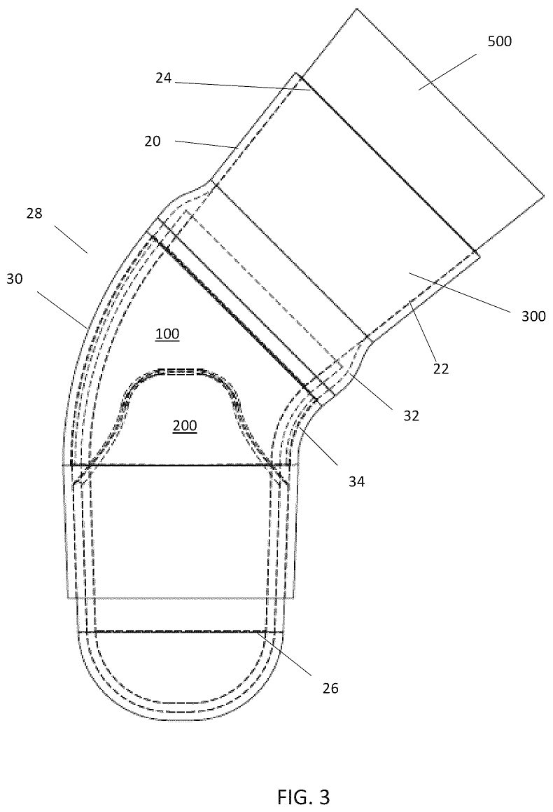

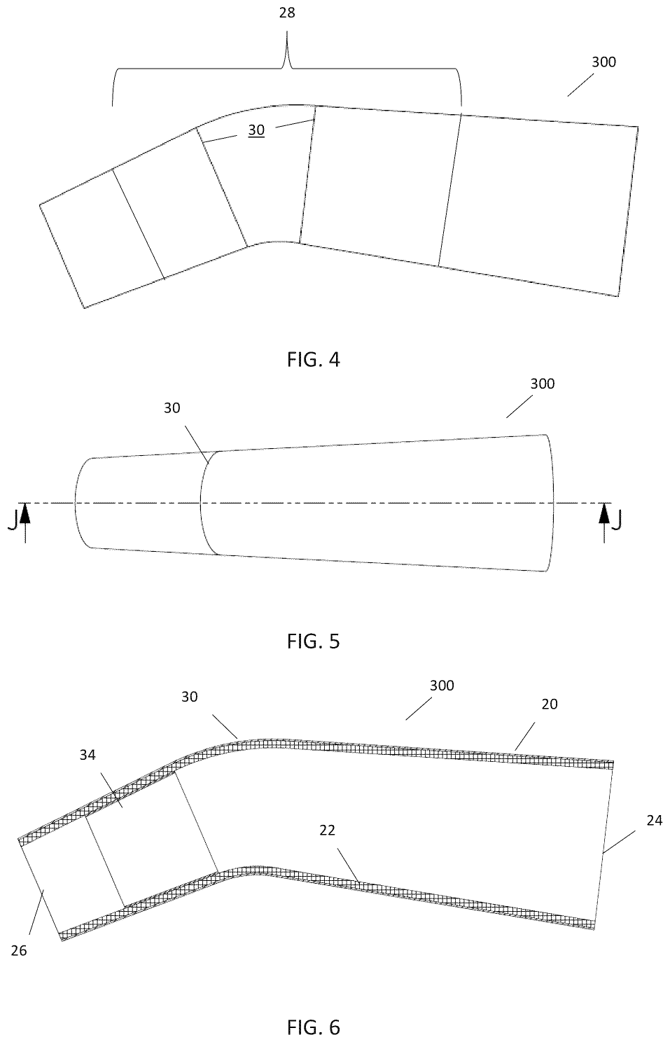

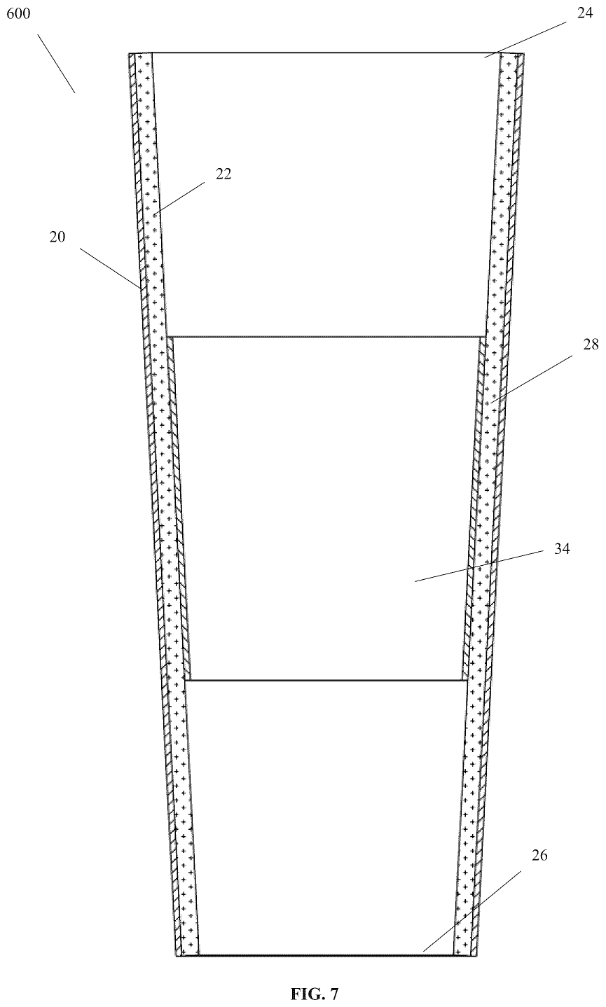

FIGS. 2 through 6 depict the pre-flexed suspension sleeve 300 of the present invention. The suspension sleeve 300 is an open-ended truncated cone with an outer fabric layer 20 and an inner cushion layer 22. Inner cushion layer 22 may be made of silicone or a thermoplastic elastomer such as a styrene triblock copolymer or other elastomer known in the art. The suspension sleeve 300 further has a proximal end 24, a distal end 26, and a midsection 28 which runs from about ⅓ of the distance from the proximal end 24 to about a ⅓ of the distance from the distal end 26, as shown in FIG. 4. The midsection 28 includes a bend 30 which ranges from 5 to 60 degrees from where the pre-flexed suspension sleeve 300 would lie if it was completely vertical. The pre-flexed suspension sleeve 300 of the present invention preferably has a bend 30 angle between 20 and 40 degrees. When under a vacuum, pre-flexed suspension sleeve 300 further forms a neck 32 which tapers towards the open proximal end 24. The inner cushion layer 22 further comprises an interior fabric reinforcement layer 34. Preferably, the interior fabric reinforcement layer 34 is located throughout the midsection 28 and may also be pre-flexed to the angle of the pre-flexed suspension sleeve 300. Alternatively, the interior fabric reinforcement layer 34 is placed below the bend 30 as that is normally where the brim 36 of the socket 200 is located when in use and therefore the area of the pre-flexed suspension sleeve 300 most susceptible to frictional forces or damage but the interior fabric reinforcement layer 34 may be located on the inner cushion layer 22 anywhere that it is likely that it will come into contact with the socket 200 once donned. For example, it may be beneficial to have the interior fabric reinforcement layer 34 located medially placed below the bend 30 for those amputees who have longer residual limbs below the knee. Alternatively, it may be beneficial to have the fabric reinforcement layer extend further above the bend 30 for those amputees who desire longer sleeves in order to extend the amount of gel above the interior fabric reinforcement layer 34 to gain an increased sense of security that the sleeve will maintain suction. In addition, the interior fabric reinforcement layer 34 may further comprise an inner elastomer layer 38. The inner elastomer 38 is preferably uniformly thick, more preferably a thickness level equivalent to individual droplets of the material use, so that the interior fabric reinforcement layer 34 can both maintain frictional contact with the socket 200 as well as with the residual limb 500. The inner elastomer layer 38 is preferably silicone but may be any elastomer or adhesive that can be applied to a uniform thickness on the interior fabric reinforcement layer 34. The inner elastomer layer 38 may be applied by spraying, rolling, brushing, blotting, or any other method that allows for uniform application. The uniform application of the inner elastomer layer 38 is important so as to maintain the stretch characteristics of the outer fabric layer 20.

The interior fabric reinforcement layer 34 of the pre-flexed suspension sleeve 300 can be attached through many methods including, but not limited to, adhesive, molding, solvent bonding, embedding and any other method known in the art. This attachment allows for a continuous polymeric material for the inner cushion layer 22 with a reinforcement that is protecting the interior surface from the socket brim 36. This reinforcement may be made of a similar or different material as the outer fabric layer 20. The fabric reinforcement layer 34 preferably has a length that is 25% of the total length of the pre-flexed suspension sleeve 300 but may be at long as the entire length of the pre-flexed suspension sleeve 300 or as short as three inches in length.

Alternatively, as shown in FIG. 7, the present invention can be a straight sleeve 600 as opposed pre-bent. The straight sleeve 600 has a slight taper from the proximal end 24 to the distal end 26. The fabric reinforcement layer 34 is preferably placed around a midsection 28 as in previous embodiments. The straight sleeve 600 has an outer fabric layer 20 and an inner cushion layer 22 having the same qualities as in previous embodiments.

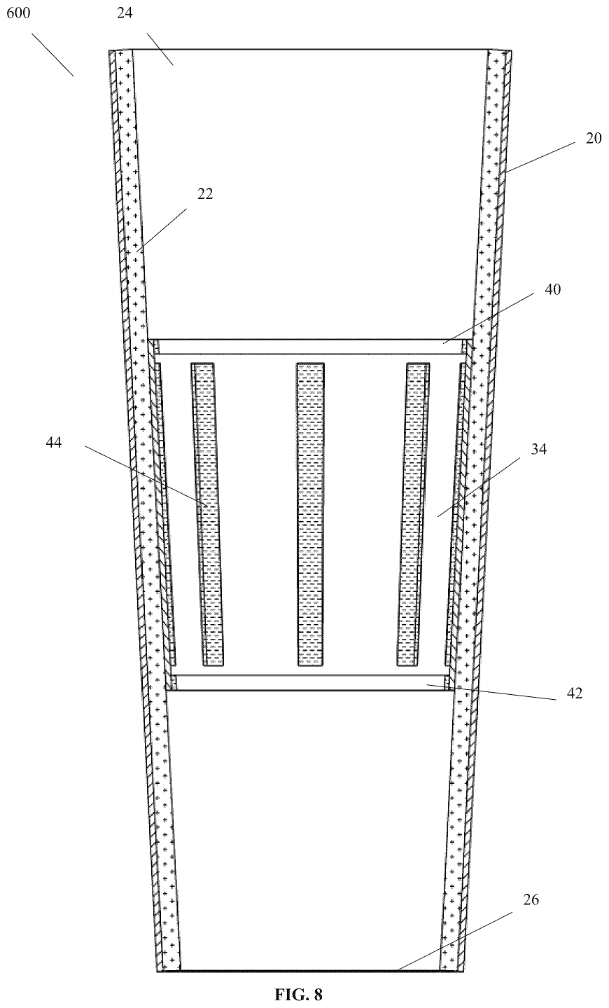

An alternative embodiment is shown in FIG. 8. Here, the fabric reinforcement layer 34 is further reinforced by a proximal circumferential band 40, a distal circumferential band 42, and multiple longitudinal retention strips 44. The proximal circumferential band 40 and the distal circumferential band 42 preferably have a thickness between 4-18 millimeters. The longitudinal retention strips 44 preferably have a thickness between 2-18 millimeters and a length that is preferably coextensive with the length of the fabric reinforcement layer 34. These additions allow for additional retention capability, an additional layer to prevent the socket from chafing the liner, and improving grip while donning and doffing the straight sleeve 600, especially for those patients with impaired dexterity. The proximal circumferential band 40, distal circumferential band 42, and longitudinal retention strips 44 are preferably made out of silicone but can also be made out of any thermoformable material that allows for rolling, brushing, or spraying onto the surface of the fabric reinforcement layer 34 during production.

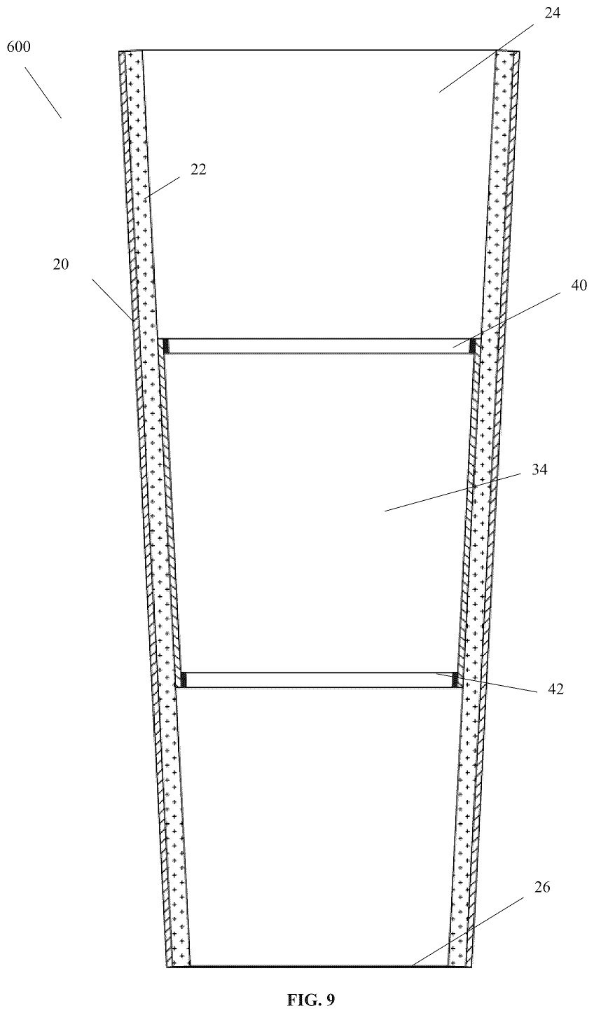

Alternatively, as shown in FIG. 9, the straight sleeve 600 may only incorporate a proximal circumferential band 40 and distal circumferential band 42 as part of the fabric reinforcement layer 34. This allows for improved sealing against the socket 200 and prevention of migration by increasing adhesion to prevent excessive movement on the socket wall or patient limb.

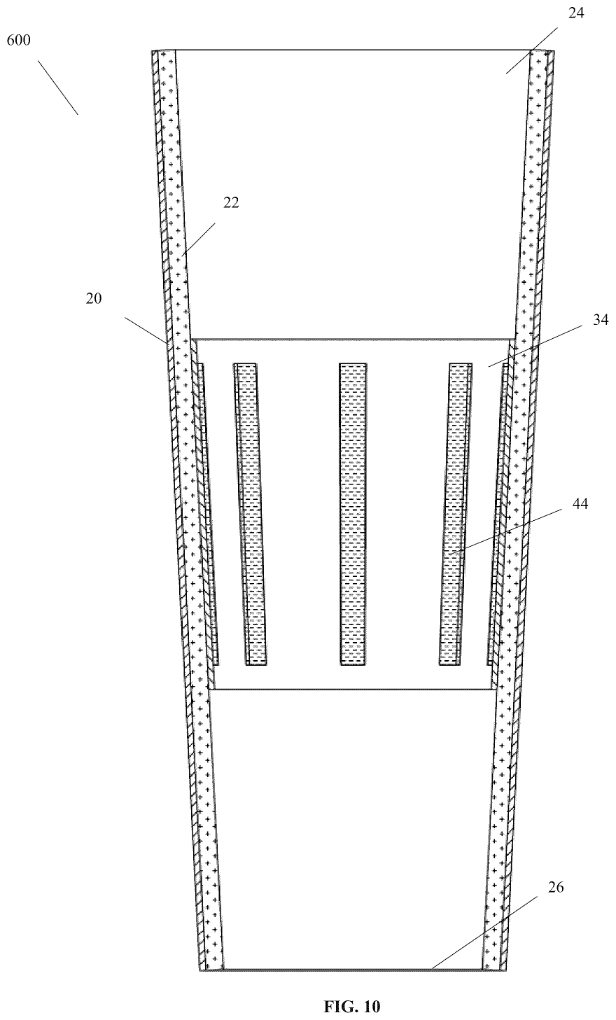

FIG. 10 shows another alternative embodiment of straight sleeve 600 wherein the fabric reinforcement layer 34 only incorporates the longitudinal retention strips 44 and does not include the proximal circumferential band 40 or distal circumferential band 42. This embodiment carries the benefit of increased suspension and donning capabilities.

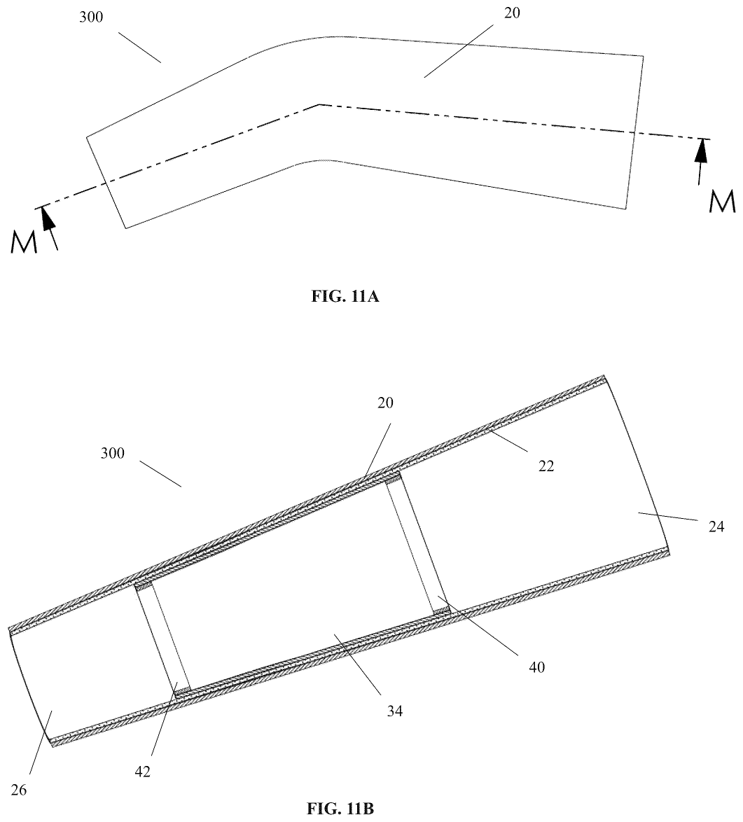

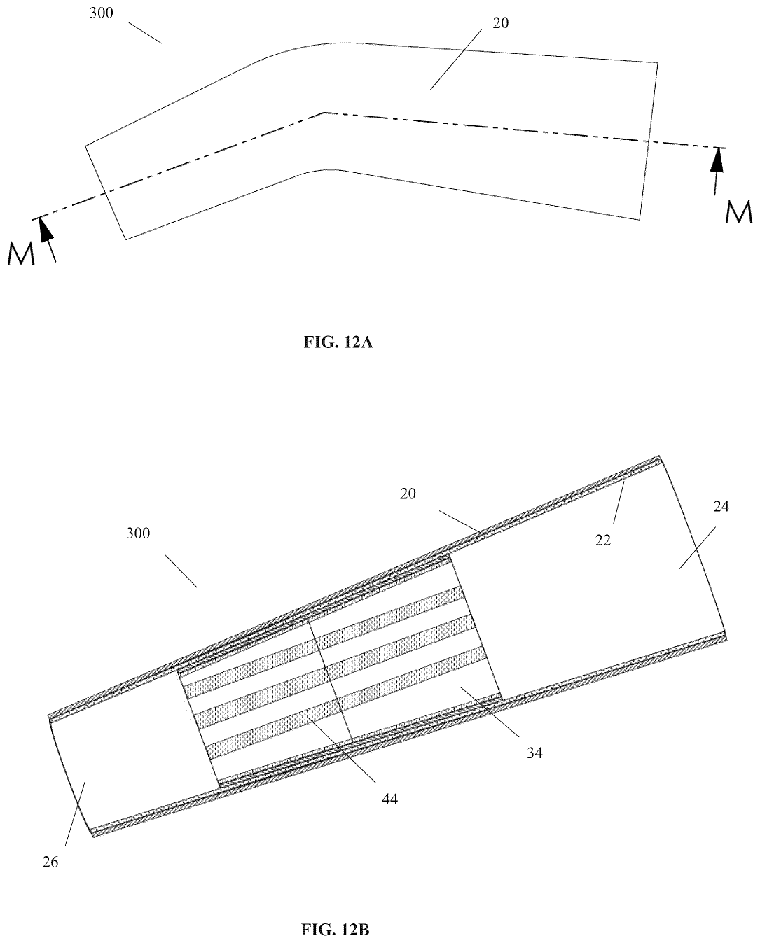

As shown in FIGS. 11A-12B, the pre-flexed sleeve 300 may have the proximal circumferential band 40 and distal circumferential band 42, as seen in FIG. 11B, or it may have the longitudinal retention strips 44, as seen in FIG. 12B. The benefits of incorporating these additions into the pre-flexed suspension sleeve 300 are the same as described in FIGS. 7-10.

The present disclosure includes that contained in the appended claims, as well as that of the foregoing description. Although this invention has been described in its preferred form with a certain degree of particularity, it is understood that the present disclosure of the preferred form has been made only by way of example and that numerous changes in the details of construction and the combination and arrangement of parts may be resorted to without departing from the spirit and scope of the invention.

Now that the invention has been described,

What is claimed is:

1. A fabric reinforced suspension sleeve comprising:
an open proximal end;
an open distal end;
an exterior fabric layer;
an inner cushion layer further comprising a midsection bent at an angle between 5 and 60 degrees; and
a fabric reinforcement layer attached to the inner cushion layer at the midsection bent to the angle of the inner cushion layer.

2. The fabric reinforced suspension sleeve of claim 1 wherein the inner cushion layer is silicone.

3. The fabric reinforced suspension sleeve of claim 1 wherein the inner cushion layer is a thermoplastic elastomer.

4. The fabric reinforced suspension sleeve of claim 1 wherein the inner cushion layer is an elastomer comprising a styrene triblock copolymer.

5. The fabric reinforced suspension sleeve of claim 1 further comprising a proximal circumferential band and a distal circumferential band incorporated on the fabric reinforcement layer.

6. The fabric reinforced suspension sleeve of claim 1 further comprising a plurality of longitudinal retention strips incorporated on the fabric reinforcement layer.

7. The fabric reinforced suspension sleeve of claim 6 further comprising a proximal circumferential band and a distal circumferential band incorporated on the fabric reinforcement layer.

8. The fabric reinforced suspension sleeve of claim 1 wherein the fabric reinforcement layer further comprises an inner elastomer layer.

9. The fabric reinforced suspension sleeve of claim 1 wherein the fabric reinforcement layer has a length that is 25% of a total length of the fabric reinforced suspension sleeve.

10. The fabric reinforced suspension sleeve of claim 1 wherein the fabric reinforcement layer has a length of at least three inches.

11. A fabric reinforced suspension sleeve comprising:
an open proximal end;
an open distal end;
an exterior fabric layer;
an inner cushion layer further comprising a midsection; and
a fabric reinforcement layer attached to the inner cushion layer at the midsection.

12. The fabric reinforced suspension sleeve of claim 11 wherein the inner cushion layer is silicone.

13. The fabric reinforced suspension sleeve of claim 11 wherein the inner cushion layer is a thermoplastic elastomer.

14. The fabric reinforced suspension sleeve of claim 11 wherein the inner cushion layer is an elastomer comprising a styrene triblock copolymer.

15. The fabric reinforced suspension sleeve of claim 11 further comprising a proximal circumferential band and a distal circumferential band incorporated on the fabric reinforcement layer.

16. The fabric reinforced suspension sleeve of claim 11 further comprising a plurality of longitudinal retention strips incorporated on the fabric reinforcement layer.

17. The fabric reinforced suspension sleeve of claim 16 further comprising a proximal circumferential band and a distal circumferential band incorporated on the fabric reinforcement layer.

18. The fabric reinforced suspension sleeve of claim 11 wherein the fabric reinforcement layer further comprises an inner elastomer layer.

19. The fabric reinforced suspension sleeve of claim 11 wherein the fabric reinforcement layer has a length that is 25% of a total length of the fabric reinforced suspension sleeve.

20. The fabric reinforced suspension sleeve of claim 11 wherein the fabric reinforcement layer has a length of at least three inches.
